(19) United States
(12) Patent Application Publication
(10) Pub. No.: US 2014/0114174 A1
(43) Pub. Date: Apr. 24, 2014

(54) SYSTEMS AND METHOD FOR QUANTIFICATION OF KNEE CARTILAGE FROM MAGNETIC RESONANCE IMAGING

(71) Applicant: TUFTS MEDICAL CENTER, Boston, MA (US)

(72) Inventors: Ming Zhang, Lowell, MA (US); Timothy McAlindon, Cambridge, MA (US); Jeffrey Driban, Boston, MA (US)

(73) Assignee: TUFTS MEDICAL CENTER, Boston, MA (US)

(21) Appl. No.: 14/060,335

(22) Filed: Oct. 22, 2013

Related U.S. Application Data
(60) Provisional application No. 61/716,757, filed on Oct. 22, 2012.

(51) Int. Cl. A61B 5/00; G01R 33/56; A61B 5/107; A61B 5/055

(52) U.S. Cl. CPC A61B 5/4514; A61B 5/055; G01R 33/5608; A61B 5/1075; A61B 5/4585
USPC 600/410

(57) ABSTRACT

The present invention relates to systems, compositions, and methods for quantification of joint cartilage from magnetic resonance imaging. In particular, the present invention relates to the use of a universal coordinate to identify informative locations in joints that reflect cartilage loss throughout the joint, reduce the burden of measuring joint cartilage, and allow for the calculation of a more sensitive measure of cartilage loss than traditional cartilage segmentation methods.

This application claims priority to U.S. Provisional Application No. 61/716,757 filed Oct. 22, 2012, which is herein incorporated by reference in its entirety.

### GOVERNMENT SUPPORT

This invention was made with government support under grant number R01 AR054938 awarded by the National Institutes of Health. The government has certain rights in the invention.

### FIELD OF THE INVENTION

The present invention relates to systems, compositions, and methods for quantification of joint cartilage from magnetic resonance imaging. In particular, the present invention relates to the use of a universal coordinate to identify informative locations in joints that reflect cartilage loss throughout the joint, reduce the burden of measuring joint cartilage, and allow for the calculation of a more sensitive measure of cartilage loss than traditional cartilage segmentation methods.

### BACKGROUND OF THE INVENTION

Osteoarthritis (OA, also known as degenerative arthritis, degenerative joint disease), is the most common form of arthritis, affecting at least 10% of the population over the age of 65, and at present there is little available in the treatment of this condition, notwithstanding NSAIDs and total joint replacements. Disability from OA is one of the leading causes of disability in the elderly. Unfortunately, the pathophysiology of this disease has not been clarified to date.

The diagnosis of OA is primarily based on history and physical examination. Usually, the clinical features that a patient exhibits—specifically the symptoms and the signs noted on examination—are sufficient to make the diagnosis of OA. To date, the most common means of confirming a diagnosis of OA is by obtaining plain radiographs of the affected joint; however, it is well-established that radiographs are notoriously insensitive to the detection of OA. Particularly because few effective treatments are available to treat this condition, identification of a measure that could predict the development of OA would be very useful. Additional methods are needed to assess early signs of osteoarthritis and to identify those who are at high risk of developing OA.

### SUMMARY OF THE INVENTION

The present invention relates to systems, compositions, and methods for quantification of joint cartilage from magnetic resonance imaging. In particular, the present invention relates to the use of a universal coordinate to identify informative locations in joints that reflect cartilage loss throughout the joint, reduce the burden of measuring joint cartilage, and allow for the calculation of a more sensitive measure of cartilage loss than traditional cartilage segmentation methods.

Embodiments of the present invention provide systems and methods for identifying informative locations rapidly and with minimal or no user input. These systems and methods decrease the amount of time and cost of analyzing imaging data, in particular large amount of data such as are encountered in clinical applications or research studies.

For example, in some embodiments, the present invention provides a method, comprising: a) applying a two-dimensional coordinate system that i) segments a joint and ii) normalizes the boundaries of the coordinate to a standardized unit of measure, to a magnetic resonance image of a joint; b) using the coordinate system to identify informative locations; and c) determining cartilage information for the informative locations. Further embodiments provide systems comprising a computer processor and computer software configured to apply a two-dimensional coordinate system to a magnetic resonance image of a joint; and i) segments the joint and ii) normalizes the boundaries of the coordinate to a standardized unit of measure to identify a informative locations and determine cartilage information for the informative locations. In some embodiments, the joint is a knee, hip, elbow, ankle, toe, finger, or other joint. In some embodiments, the informative locations define a region of the joint that is universal across multiple joints. In some embodiments, the cartilage information comprises a measure of osteoarthritis in the joint. In some embodiments, the applying comprises the step of identifying the boundary of the joint. In some embodiments, the boundary is determined by a user or by the system.

Additional embodiments are described herein.

### DESCRIPTION OF THE FIGURE

FIG. 1 shows a flow chart of systems and methods of embodiments of the present disclosure.

FIG. 2 shows points of femur segmentation.

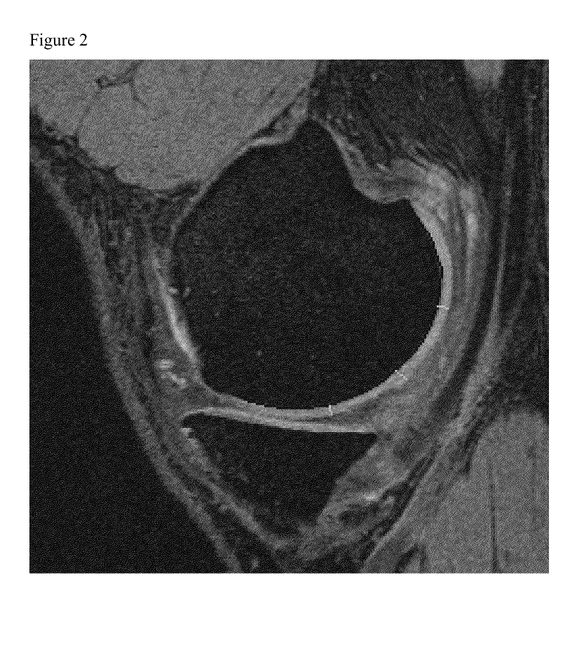

FIG. 3 shows points of tibia segmentation.

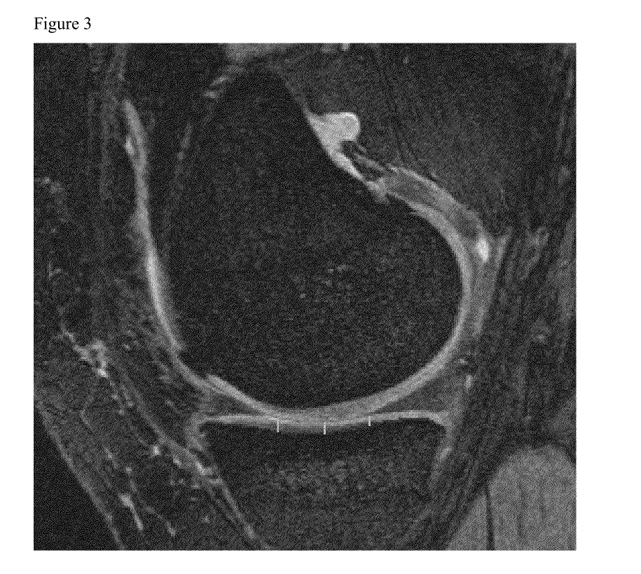

### DEFINITIONS

As used herein, the term “substantially” refers to greater than 75% (e.g., greater than 80%, 85%, 90%, 95%, 98%, or 99%).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “informative locations” refers to regions (e.g., regions of a joint) that provide information. For example, in some embodiments, informative locations are regions of a joint that provide cartilage information (e.g., cartilage information useful in diagnostic, screening, or research uses). In some embodiments, informative locations are regions of an image (e.g., MR image) of a joint.

As used herein, the term “a subject diagnosed with osteoarthritis in a joint” refers to a subject that has been diagnosed with osteoarthritis based on one or more diagnostic assessments (e.g., magnetic resonance imaging (MRI) of the joint, x-ray, physical examination, etc.)

### DETAILED DESCRIPTION OF THE INVENTION

The present invention relates to systems, compositions, and methods for quantification of joint cartilage from magnetic resonance imaging. In particular, the present invention relates to the use of a universal coordinate to identify informative locations in joints that reflect cartilage loss throughout the joint, reduce the burden of measuring joint cartilage, and allow for the calculation of a more sensitive measure of cartilage loss than traditional cartilage segmentation methods.

In some embodiments, the present disclosure provides systems and methods for quantifying human joint (e.g., knee) cartilage in magnetic resonance (MR) images. In some embodiments, the levels of cartilage are used to diagnose, monitor, or asses osteoarthritis (OA) or OA treatments. The systems and methods described herein automate and standardize the identification and quantification of informative locations and thus reduce manual segmentation time.

Knee OA is the most common form of arthritis, accounting to substantial disability in the general population (Lawrence et al., Arthritis and Rheumatism 2008; 58:26-35). A common characteristic of OA is articular cartilage loss. However, obtaining accurate and reproducible quantitative cartilage information from MR images is still an open problem. Cartilage measures (e.g., volume, thickness, surface area, curvature) require the cartilage to be segmented (Cohen et al., Osteoarthritis Cartilage 1999; 7:95-109; Stammberger et al., Magnetic Resonance in Medicine 1999; 41:529-36; Hohe et al., Magnetic Resonance in Medicine 2002; 47:554-61), which can be a time consuming process. In addition to the time required to segment the knees, it can take three months of training for operators, who segment the images, to become accurate and reliable (JL J et al., Osteoarthritis Cartilage 2006; 14:914-22). Even with a well-trained individual segmenting the images, a registration process is needed for longitudinal studies to account for the variation of MR knee scan position or joint angle. Some computer-aided algorithms are designed to help this process, but none of them can be efficiently used in large clinical trials (Eckstein et al., Arthritis 2011; 2011:1-19).

Over the past decade, researchers have deployed several approaches to try and reduce the burden of segmenting knee cartilage on MR images. Some researchers choose to segment every other MR image, which only reduces half of the work. Other existing methods are used to look at the OA pattern within whole or partial regions of cartilage (Steines D, U.S. Pat. No. 6,799,066, 2004). The segmentation time is still high. Finally, some researchers have developed automated cartilage segmentation algorithms (e.g., active contour, B-spline, etc.) (Cashman et al., IEEE Transactions on NanoBioscience 2002; 1:42-51; Tameem et al., In: AIP Conference Proceedings, vol. 953 2007:262-76) but they don't provide accurate or reliable data (Eckstein et al., Arthritis 2011; 2011:1-19).

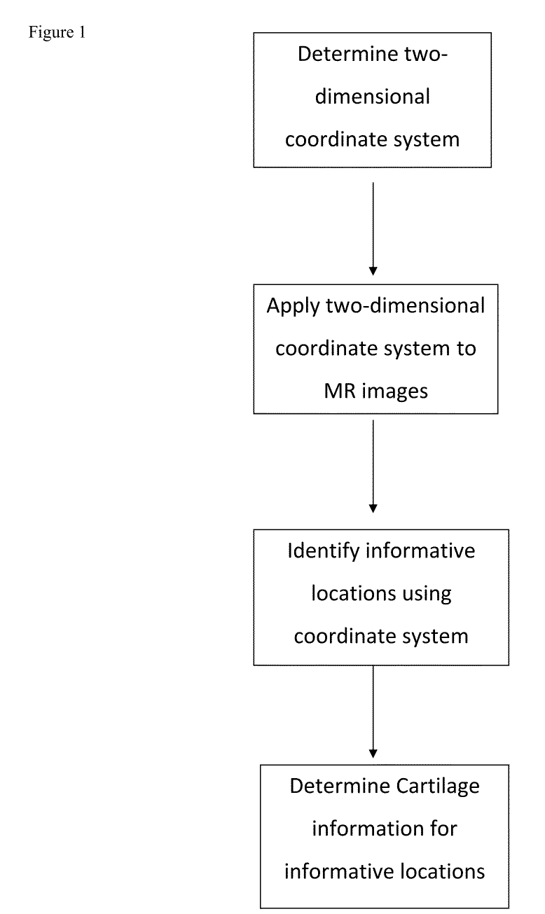

Embodiments of the present disclosure solve several of the problems associated with standard methods of generating cartilage data. For example, embodiments of the present disclosure provide a universal coordinate system for all knees; which functions as an efficient registration process. Furthermore, this coordinate system can locate informative locations that are representative of cartilage loss in the knee. By only focusing on the informative locations, the segmentation time is reduced to several minutes per knee. FIG. 1 shows an overview of systems and method of embodiments of the present disclosure.

In some embodiments, the present invention provides an informative-location (e.g., specific anatomical points of interest) based method rather than regions-of-interest or whole cartilage method. This approach greatly reduces the manual interaction time. In addition, the segmentation time based on the methods described herein is constant even among data sets with different MR scan resolution or number of slices. Furthermore, the systems and methods described herein simplify the cartilage segmentation procedure. No image registration is needed, which saves a lot of manual processing time. The user only needs to identify the first and last slice of the MR sequence which contains the joint and then outline the knee bones in a few slices.

Accordingly, in some embodiments, the present invention provides systems and methods that find use in analyzing large amounts of MR image data set, which is nearly impossible or impractical for the traditional and existing methods.

This present disclosure comprises, in some embodiments, three major components for performing the above functions: 1) universal coordinate systems for joints, 2) cartilage thickness measurements at informative locations of a joint, and 3) calculation of cartilage parameters in a joint.

The present is not limited to a particular joint. The systems and methods described herein find use in the analysis of a variety of joints. While the present invention is exemplified with knee joints, the present invention finds use in the analysis of a variety of joints (e.g., including but not limited to, knee, hip, elbow, ankle, etc.).

In some embodiments, the present disclosure provides a universal coordinate systems for knees to identify regions or points of interest (ROI or POI). In some embodiments, two, 2-dimensional rectangular coordinate systems are used to represent the articular surfaces of a joint (e.g., proximal tibia and distal femur in the knee). Within the coordinate system, the vertical axis represents the medial-to-lateral width of the joint (e.g., sagittal MR image slice sequence number) and the horizontal axis represents the anterior-posterior length of the articular surface. In some embodiments, to account for variations across joint sizes and MR image sets (e.g., image resolution) the coordinate system is standardized so that the vertical and horizontal axes have a standardized reference length. The reference length may be measured using any suitable unit of measurement. In some embodiments, MR images are measured in voxels, although other units may be utilized. The reference length is chosen to be universally applicable across subjects (e.g., multiple joint sizes) and provide consistent information. In some experimental embodiments described herein, the reference length was 200 voxels on the vertical axis 500 voxels on the horizontal axis, although other sizes are suitable.

This approach provides a robust method to identify specific locations within a joint regardless of joint size or reference length size. For example, if the width of joint A is 50 slices (e.g., 50 voxels) and the width of joint B is 100 slices (e.g., 100 voxels) and the image of interest is located on the universal coordinate system at the twentieth voxel on the vertical axis then the coordinate system recognizes that slice 5 and 10 are anatomically the same slice of interest; in joints A and B respectively. Similarly, regardless of joint size the systems and methods described herein can find specific cartilage locations along the anterior-posterior length of an articular surface using the horizontal axis. Experiments conducted during the course of development of embodiments of the present invention tested the universal coordinate by using approximately 170 knees from a cohort study (Osteoarthritis Initiative) and a clinical trial.

In some embodiments, prior to application of the systems and methods described herein, the appropriate location(s) of informative locations on the universal coordinate system are determined for a particular joint. In some embodiments, the informative locations on the coordinate system of are determined based on manual or automated examination of images of joints. For example, in some embodiments, a plurality (e.g., 20, 30, 50, 100 or more) images of different sized joints from a plurality (e.g., 20, 30, 50, 100 or more) individuals are compared to identify where on the universal coordinate the informative locations are located. This process is repeated for different joints.

The informative locations are selected based on any suitable criteria. In some embodiments, informative locations are selected based on preliminary testing in datasets. For example, in experiments conducted during the course of development of embodiments of the present disclosure, two clinical datasets were used to evaluate the relationship between cartilage parameters calculated with the informative location approach and whole cartilage segmentation. Furthermore, the informative locations were selected based on analyses of the responsiveness to detect change (Hunter et al., Osteoarthritis Cartilage 2006; 14:112-15) when using cartilage parameters calculated with this informative location approach or whole cartilage segmentation.

To orient an MR image set of a joint to the universal coordinate system, a user or computer software indicates the most medial and lateral MR slices that represent the joint. After the width of the joint is established the coordinate system automatically indicates MR slices that contain informative locations. The bone-cartilage boundary is then marked manually or using computer software and a computer processor. Once the bone-cartilage boundary is marked, software converts the length of the articular surface to the standardized horizontal axis.

In some embodiments, the present disclosure further provides systems and methods for measuring the articular cartilage thickness on MR images by using the predefined informative locations. These informative locations are identified after an MR image set is oriented on the universal coordinate system. The systems and methods described herein mark the POI to facilitate quick measurements. Unlike other existing methods, the systems and methods described herein do not require a reader to segment the whole cartilage or bone to apply the data to a coordinate system.

In some embodiments, the present disclosure further provides systems and methods for calculating cartilage parameters (e.g., cartilage thickness, estimated cartilage volume, changes in cartilage parameters) based on the informative locations. In some embodiments, the cartilage parameters are used to diagnose osteoarthritis (OA), risk of developing OA in a joint, progression of OA, or to assess the effect of a therapy for OA.

In some embodiments, a system is provided comprising imaging devices and software for performing the operations described herein. In some embodiments, software is incorporated into the imaging device (e.g., on a computer processor attached to the imaging device).

In some embodiments, the system provides an image of the joint to be analyzed and the user (e.g., clinician) uses the software to identify the informative locations for analyzing cartilage. In some embodiments, the software then calculates cartilage data for the informative locations identified using the system.

In some embodiments, data analysis software provides information in a format that is useful for a clinician without further analysis. For example, in some embodiments, one or more measurements of cartilage or joint health or level of osteoarthritis is provided. In some embodiments, a representation (e.g., graphical) of the joint health of a given subject over time are provided. In some embodiments, the data analysis software provides a quantitative (e.g., probability) or qualitative assessment of the risk of developing osteoarthritis or the risk of progression of existing osteoarthritis based on the parameters measured.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method, comprising:
a) applying a two-dimensional coordinate system that i) segments a joint and ii) normalizes the boundaries of the coordinate to a standardized unit of measure, to a magnetic resonance image of a joint;
b) using said coordinate system to identify informative locations; and
c) determining cartilage information based on said informative locations.

2. The method of claim 1, wherein said joint is a knee joint.

3. The method of claim 1, wherein said informative locations defines locations of said joint that is universal across multiple joints.

4. The method of claim 1, wherein said cartilage information comprises a measure of osteoarthritis or cartilage loss in said joint.

5. The method of claim 1, wherein said applying comprises the step of identifying the boundary of said joint.

6. The method of claim 5, wherein said boundary is determined by a user or by said system.

7. The method of claim 1, wherein said system comprises a computer processor and software for performing said method.

8. A system, comprising:
A computer processor and computer software configured to apply a two-dimensional coordinate system to a magnetic resonance image of a joint; and i) segments said joint and ii) normalizes the boundaries of the coordinate to a standardized unit of measure to identify informative locations and determine cartilage information based on said locations.

9. The system of claim 8, wherein said informative locations define locations of said joint that is universal across multiple joints.

10. The system of claim 8, wherein said cartilage information comprises a measure of osteoarthritis or cartilage loss in said joint.

11. The system of claim 8, wherein said applying comprises the step of identifying the boundary of said joint.

12. The system of claim 11, wherein said boundary is determined by a user or by said system.
